(19) United States
(12) Patent Application Publication
(10) Pub. No.: US 2007/0238163 A1
(43) Pub. Date: Oct. 11, 2007

(54) Covalent attachment of biomolecules to solid supports by a polymerization method

(76) Inventors: Julia B. Golova, Burr Ridge, IL (US); Boris Chernov, Burr Ridge, IL (US); Alexander Kukhtin, Lockport, IL (US)

Correspondence Address: BARNES & THORNBURG LLP, P.O. BOX 2786, CHICAGO, IL, 60690-2786, US

(21) Appl. No.: 11/399,034

(22) Filed: Apr. 5, 2006

(51) Int. Cl. C12M 1/34; G01N 1/28

(52) U.S. Cl. 435/287.2; 427/2.11; 977/902

(57) ABSTRACT

Described herein is a novel approach for the immobilization of one or more biomolecules or biomolecule complexes to solid supports. The method describes the modification of a solid support, if required, attaching functionality to a biomolecule or biomolecule complex, and linking the biomolecule or biomolecule complex to the solid support. The described complexes are rapidly produced while conserving both orientation and functional activity of the utilized biomolecule or biomolecule complex. Thus, materials produced by this method thus have applications as nanomaterials for biosensor fabrication, screening microarrays, high-throughput drug discovery, and as a tool for combinatorial chemistry, to name only a few.

### BACKGROUND

Developing novel approaches to immobilize one or more biomolecules or biomolecule complexes to various solid supports is the focus of numerous areas within the physical and material sciences (e.g., microarray manufacturing, material engineering, and molecular biological applications). Within these fields, the preservation of the activity of the immobilized biomolecules is required so the materials can be ultimately utilized as nanomaterials for biosensor fabrication, in the manufacture of screening microarrays for diagnostic assays, for high-throughput drug discovery, and as a tool for combinatorial chemistry, for example.

The covalent attachment of biomolecules to various solid supports is currently a strategy for immobilization. This method provides a stable connection through a covalent bond between an immobilized probe and a solid support. This approach utilizes different active chemical groups which provide stable covalent bonds between the probe and support. The covalent bond between the solid support and biomolecule can be formed by utilizing functional groups already present on the biomolecule, for example amino, thio, and carboxylate. However, immobilizing biomolecules to the solid support through these groups on the molecule can lead to partial, or even complete, loss of the activity of the attached biomolecule. This significantly alters or prevents the chemically selective coupling of probes to the support.

Attempts to overcome this problem and conserve orientation and functional activity of the biomolecules include chemically affixing active groups to the immobilized probe and support. The specific functional groups used for chemo-selective immobilization can be introduced into synthetic DNA fragments, peptides or carbohydrates during their chemical synthesis. For fragments of natural DNA or other biomolecules (like proteins, antibodies, receptors and the like) special modifier reagents should be used for incorporation of functional groups able to provide chemo-selective immobilization. Also, preliminary functionalization of solid supports should be performed for successful immobilization. However, no single method provides for an optimally effective immobilized biological probe. Usually, incorporation of such active groups requires a multistep chemical treatment both of a support and a biomolecule. Furthermore, when incorporated, the covalent coupling between the biomolecule and the ligand provides a sterically disfavored environment (especially for proteins, antibodies, receptors) thereby limiting the complex's use in affinity interactions. Moreover, most of the methods employed are time-consuming and applicable for only a limited number of supports.

### SUMMARY

A versatile approach for the chemo-selective graft immobilization of a wide range of biomolecules (e.g., synthetic and natural DNA fragments, peptides, proteins, carbohydrates) to practically any solid support system (e.g., glass, plastics, metal, silica surfaces; hydrogel substrates; carbon nanotubes; nanoparticles and others) is by modifying the solid support and the biomolecule with acrylate functional groups followed by their chemo selective interaction. The solid support and biomolecule are then covalently bonded together via graft polymerization. The afforded complex has many uses, including: the manufacture of screening microarrays, biosensors producing, diagnostic assays, high-throughput drug discovery, and as a tool for combinatorial chemistry.

This method for graft-immobilizing a biomolecule on a solid support includes the steps of:

(a) providing a solid support comprising a first acrylate functional group;

(b) covalently attaching a second acrylate functional group to the biomolecule; and

(c) linking the first acrylate functional group to the second acrylate functional group with a covalent bond.

Alternatively, the modification of the solid support (step a) utilizes a first methacrylate functional group.

In an alternative embodiment, the modification of a biomolecules (step b) can utilize a second methacrylate functional group.

It to be understood that as used herein, the term acrylate refers both to substituted and unsubstituted acrylic acids. Illustratively, acrylates include, but are not limited to, acrylic acids, methacrylic acids, cyanoacrylic acids, crotonic acids, maleic acids, fumaric acids, itaconic acids, citraconic acids, mesaconic acids, and the like. In addition, it is to be understood that as used herein, acrylate starting materials that are used to prepare the compounds described herein may be anhydrides, chloroanhydrides or activated esters.

The solid support utilized may be selected from, but is not limited to, glass, quartz, plastics, silicon, gold, hydrogels, carbon nano tubes, and nanoparticles.

Incorporation of the first acrylate functional group into solid supports containing amino groups is carried out by using a compound of the formula:

H₂C═C(R¹)CO—X; wherein R¹ is hydrogen, alkyl, or cyano; and X is a leaving group. This leaving group can be selected from the group including N-hydroxysuccinimide and its analogs, halides, and carboxylates (for example, OCO(CH₃)C═CH₂).

The first acrylate functional group can also be introduced into aminated solid supports by using a compound of the formula:

H₂C═C(R¹)CO—Y-Q-Y-Z; wherein R¹ is hydrogen, alkyl, or cyano; Y=—NH— or —O—; Q is a branched, linear, or cyclic alkylene group; and Z is an activated carboxylic acid (e.g., COIm or CH₂COONHS). Without being bound by theory, it is suggested and appreciated that as the size of the alkylene group, and thus total number of carbon atoms, is either reduced or augmented, potential intra- or intermolecular steric interactions or solvent effects may mediate the activity of the linked biomolecule and solid support. Thus, in one illustrative embodiment, the minimum number of carbon atoms in the alkylene group is 6, a value that may reduce steric interactions that would otherwise decrease interactions of the biomolecule with an analyte of interest. In another illustrative embodiment, the maximum number of carbon atoms in the alkylene group may depend on the selection of the solvent and biomolecule. As illustrative examples, the alkylene (Q) may be of the formula (CH₂)ₙ, where n=4-12, or a poly(alkylene oxide), such as PEG₄₀₀₋₆₀₀₀.

Providing a solid support, such as a silicon-based support, with a methylacrylate group functionality is carried out by treating the support with a compound of the formula:
H₂C═CH—R—NH₂; wherein R is an alkylene moiety selected from the group consisting of (CH₂)ₙ, where n=4-12, and PEG₄₀₀₋₆₀₀₀.

The resulting aminated solid support is further reacted with an acrylating reagent of the formula:
H₂C═C(CH₃)CO—X; wherein X is a moiety selected from the group consisting of N-hydroxysuccinimide and analogs and derivatives thereof, halo, and carboxylates.

In another embodiment, the aminated hydrogel solid support can be obtained by further reacting it with an acrylating reagent of the formula:
H₂C═C(R¹)—CONH-Q-NH₂; where R¹ is H or CH₃; and Q is selected from (CH₂)ₙ, n=4-12, and PEG₄₀₀₋₆₀₀₀.

This method further includes the reaction of resulting aminated hydrogel support with a methacrylating reagent of the general formula:
H₂C═C(CH₃)CO—X; wherein X is N-hydroxysuccinimide and analogs, halo, and carboxylates.

In another embodiment, the solid support includes gold, to which an acrylate group is incorporated by treating the support first with a compound of the formula:
R₁S—(CH₂)ₙ—NH₂, where n=4-12; and R¹ is selected from the group consisting of H, alkyl C₂-C₆—S, or disulfides.

The resulting aminated gold support is subsequently treated with a methacrylating reagent of the formula:
H₂C═C(CH₃)CO—X; where X is such leaving groups as NOS, Cl, Br, OCO(CH₃)C═CH₂.

The biomolecules used in the methods described herein also possesses an acrylate group, either originally, or by the addition of an acrylate or methylacrylic functional group as described herein. In one embodiment, the biomolecule or biomolecule complex is selected from the group consisting of synthetic oligonucleotides or fragments of natural nucleic acids, synthetic or natural peptides, proteins and carbohydrates.

In one variation of this embodiment, the biomolecule is a synthetic oligonucotide modified by reacting it with acrylating reagents of the formula: 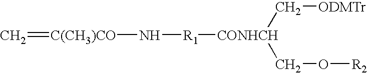
wherein
R₁=(CH₂)ₙ, n=2-12; R₂=P(OCH₂CH₂CN)N(iPr)₂ or CO(CH₂)ₘCO-CPG where m=1-8.

The oligonucleotide biomolecule may be modified after it is obtained, or the modification may be made during the synthesis using conventional automated syntheses procedures wherein one or more steps includes the acrylating agents described herein.

In another embodiment, biomolecules comprising peptides are synthesized or modified to include acrylate functionalities by reaction with compounds of the formula:
H₂C═C(CH₃)CONH—(CH₂)ₙ—CONHS; where n=2-12.

The peptide biomolecule may be modified after it is obtained, or the modification may be made during the synthesis using conventional automated syntheses procedures wherein one or more steps include the acrylating agents described herein.

The method includes one or more biomolecules or a biomolecule complex. The single biomolecule or biomolecular complex comprising proteins is modified by treating it with an acrylating regent of the formula:
H₂C═C(CH₃)CONH—(CH₂)ₙ—C₆H₄—COCH₂Br; where n=2-6.
or
H₂C═C(CH₃)CONH—R₃—NHCOCH₂—X; where X═Br, I; R₃=alkylene, C₂-C₁₂, or cycloalkylene C₃-C₈.

The attachment step includes covalent bond formation through graft polymerization initiated by UV exposure or by using a radical initiator.

A microarray comprising the reaction products of:
(a) a solid support comprising a first acrylate group; and
(b) a biomolecule comprising a second acrylate group;

where the reaction includes the attachment of one or more biomolecules to a solid support.

Definitions and Abbreviations

Biomolecule—any organic molecule that was, or could be, an essential part of a living organism such as DNA fragments, peptides, proteins, lipids, affinity ligands, and the like.

Biomolecule Complex—An array of biomolecule complexes such as ribonucleic acids, fragments of DNA or RNA, peptides, proteins, lipids, and tissues; these complexes can be multiples of biomolecules, and/or biomolecules that are ligated and/or modified.

CPG—control pore glass or controlled pore glass.

DMTr—4,4′-dimethoxytrityl protecting group.

DNA—deoxyribonucleic acid

FG—functional group.

HPLC—high pressure liquid chromatography.
Hydrogel—a colloidal gel in which water is the dispersion medium.

Im—imidazolyl.

Linker: a polyfunctional molecule containing functional groups that may connect different parts of a chemical structure.

MALDI-TOF MS—matrix-assisted laser desorption/ionization time-of-flight mass spectrometry.

Microarray: a predetermined arrangement of molecules relative to each other connected to a support, also referred to as a chip, DNA chip, DNA microarray, DNA array, microchip, peptide chip, or peptide array.

Modifier: a chemical that provides at least one functional amino group to a molecule, illustratively to an oligonucleotide.

MW—molecular weight.

Nanoscience—primarily the extension of existing sciences into the realms of the extremely small (nanomaterials, nanochemistry, nanobio, nanophysics and the like); it concerns itself with the study of objects which are anywhere from hundreds to tens of nanometers in size.

NHS—N-hydroxysuccinimidyl.

Oligonucleotide: A nucleotide sequence (DNA or RNA) having about 6 or more nucleotides, and illustratively in the range from about 6 to about 100 nucleotides.

PEG—polyethylene glycol.

Phosphoramidite—phosphoramide derivatives of nucleosides used in chemical solid phase oligonucleotide synthesis.

RNA—ribonucleic acid.

Solid support—carrier for automated solid phase oligonucleotide synthesis, illustratively control pore glass, long-chain control pore glass.

TEAA—triethylammonium acetate buffer.

TLC—thin layer chromatography.

UV—ultraviolet.

### DETAILED DESCRIPTION

A method for immobilizing a biomolecule on a solid support by first providing a methylacrylic functional group on a solid support, modifying a biomolecule with a methylacrylic functional group, and then immobilizing a modified biomolecule on the solid support via graft polymerization of methylacrylic functionalities with formation of a covalent C—C bond.

The immobilization of biomolecules to a solid support is achieved by incorporation of selectively modified polymeric moieties that are capable of being added to a solid support and biological molecule, and then covalently reacted (e.g., polymerized) to afford platforms for further applications in nanomaterials, biosensor fabrication, screening microarrays, diagnostic assays, drug discovery, and combinatorial chemistry. Virtually any support currently available or synthesized in the future, for instance glass, quartz, plastics, silicon, hydrogel, carbon nano-tube, and nanoparticles, are suitable.

The acrylic, methacrylic, or derivatives moieties thereof, affixed to the solid support bearing free amino groups are introduced by using of compound of the general formula H₂C═C(R)CO—X, where R is CH₃ or H, X is an electron withdrawing group selected from the group consisting of NHS and analogs and derivatives thereof, halo, and carboxylates (e.g., OCO(CH₃)C═CH_{2.}).

Alternatively, the polymeric functional group can be incorporated into an aminated solid support by using of modifier of the general formula: H₂C═C(CH₃)CO—Y-Q-Y-Z; wherein Y=NH or O; Q is selected from the group consisting of an alkyl straight chain (CH₂)ₙ, where n=4-12, alkyl branched chain, or PEG₄₀₀₋₆₀₀₀, and Z=CO—Im or CH₂COONHS.

Incorporating the acrylic functionality on to a silicon solid support is achieved by treating the solid support with a compound of the formula H₂C═CH—R—NH₂; wherein R is an alkyl moiety selected from the group consisting of alkyl (CH₂)ₙ, n=4-12 and PEG₄₀₀₋₆₀₀₀ Subsequently added to react with the amine functionality is an acrylating reagent of the formula H₂C═C(R)CO—X; wherein R is H or CH₃, X is a NHS, Cl, Br, or OCO(CH₃)C═CH₂ moiety.

Introducing of acrylic or methacrylic functions into a hydrogel solid support is achieved by two step procedure: at first the gel forming mixture is copolymerized with monomers of the formula: H₂C═C—R₁—CONH—R₂—NH₂; where R₁ is H or CH₃, and the length of R₂ can be selectively chosen to be a straight chain (CH₂)ₙ, n=4-12, alkene, branched alkyl carbon chains, or any combination thereof; R₂ can also be PEG₄₀₀₋₆₀₀₀ Then this aminated hydrogel support is allowed to react with methacrylating reagent having the general formula: H₂C═C(R)CO—X; wherein CH₃ or H, X is NHS and analogs and derivatives thereof, halo, carboxylates (e.g., OCO(CH₃)C═CH_{2.}), or NOS.

Modification of a gold solid support is achieved by sequential treatment with a compound of the formula: R₁S—(CH₂)ₙ—NH₂, where n=4-12; R₁ is selected from the group consisting of H or alkyl C₂-C₆—S following by applying of a methacrylating reagent having the formula: H₂C═C(CH₃)CO—X; X is NHS and analogs and derivatives thereof, halogens, carboxylates (e.g., OCO(CH₃)C═CH_{2.}), or NOS.

A biomolecule (e.g., proteins, nucleic acids, peptides, and carbohydrates) or biomolecule complex is modified through an incorporation of a polymerizable functional group (e.g., acryloyl, methacryloyl, or derivatives thereof). There are several approaches for incorporation of polymerizable fulctional groups into different kinds of biomolecules.

For the modification of synthetic DNA fragments in a process of automated solid phase oligonucleotide synthesis, a compound of the general formula is used:

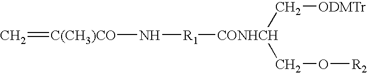

wherein R₁=(CH₂)n, n=2-12; R₂=P(OCH₂CH₂CN)N(iPr)₂ or CO(CH₂)mCO-CPG where m =1-8.

Alternatively, a biomolecule represented by synthetic peptide can be modified through an attachment on the last step of synthesis of a polymerizable functionality by using a modifier of the formula:
H₂C═C(CH₃)CONH—(CH₂)ₙ—CONHS; where n=2-12.

Additionally, a single biomolecule or a biomolecule complex displayed as proteins can be modified through the incorporation of polymerizable functionality by treatment with compound of the formula:
H₂C═C(CH₃)CONH—(CH₂)ₙ—C₆H₄—COCH₂Br; where n=2-6.
or
H₂C═C(CH₃)CONH—R₃—NHCOCH₂—X; where X=Br, I; R₃=Alkylene, C₂-C₁₂, or cycloalkene C₃-C₈.

In one illustrative embodiment, the covalent attachment of the biomolecule or biomolecule complex to the solid support may be prepared via graft polymerization. The process may be initiated by chemical or irradiative (for example, ultraviolet) sources and permits polymerization between unsaturated functional groups on the solid support and biomolecule.

Materials and Methods

1. Synthesis of 6-(Methacryloylamido) hexylamine (Compound I)

1,6-diaminohexane (1.89 g, 15 mmol) was dried by co-evaporation with pyridine (2×30 ml) and dissolved in 60 ml of dry pyridine. 4-Methoxytriryl chloride (3.08 g, 10 mmol) was added to this solution; the mixture was stirred at room temperature for 4 h, and concentrated in a vacuum. The resulting oil was dissolved in chloroform, washed with 5% aq. NaHCO₃, water, dried over Na₂SO₄.The solvent was removed in a vacuum, and crude product was dissolved in acetonitrile (50 ml). N-hydroxysuccinimidyl methacrylate (1.83 g, 10 mmol) in 25 ml of acetonitrile was added to this solution, and the mixture was stirred at room temperature for 2 h. The reaction was monitored by TLC (silica gel 60 F₂₅₄, hexane : acetone=1: 1). After the reaction was completed the mixture was concentrated in a vacuum and partitioned between ethylacetate and 5% aq. NaHCO₃. The organic layer was washed with water, concentrated to a syrupy mass, then 50 ml of 80% acetic acid was added, and solution was stirred at 50° C. for 30 min. All volatiles were removed in vacuum, the oil obtained was partitioned between water and ether aqueous part was adjusted to pH 8-9 using 1 M NaOH and then extracted with chloroform : n-butanone (5:1). Volatile solvents were removed under vacuum, and resulting product (Compound I) (1.5 g, yield, 83%) was used for further reaction without additional purification. MALDI MS calc. MW for C_{1O}H_{2O}N₂0=184.23; found (M) 184.22.

N-(Hydroxysuccinimidyl)methacrylate (Compound II)

A solution of methacryloyl chloride (3.12 g, 30 mmol) in 20 ml of tetrahydrofuran was added dropwise at 0° C. under stirring to the mixture of N-hydroxysuccinimide (3.45 g, 30 mmol) and triethylamine (4.2 ml, 30 mmol) in 150 ml of tetrahydrofuran. Stirring was continued for 1 hour, and then precipitate of triethylamine-hydrochloric acid was removed by filtration. The filtrate was evaporated in a vacuum, the resulting material was partitioned between water and chloroform, and the organic phase was dried over Na₂SO₄ and concentrated to dryness in a vacuum. The obtained product was crystallized from methanol to afford 4.2 g (76%) of Compound II. MALDI MS: calc. MW for C₈H₉N0₄=183.04; found (M) 183.09.

Synthesis of 5′-methacrylated oligonucleotides

Oligonucleotide synthesis was carried out on an AB 394 DNA/RNA synthesizer (Applied Biosystems US) in 1 umol scale using phosphoramidite chemistry. The cleavage of oligonucleotide from the CPG and removing of protecting groups were performed following the standard procedure. After HPLC purification the products were characterized by MALDI-TOF MS.

Fabrication of Polyacrylamide Gel Array Template

A polymerization chamber consisted of a glass slide treated with Bind-Silane (AmershamPharmacia-Biotech, Piscataway, N.J.) and a quartz plate mask with the specifications: transparent, 100×100 μm square windows, spaced by 200 μm, was arranged on a 1 pm thick chromium non- transparent layer. 10 tm-thick spacers separated the slide and the mask.

The polymerization mix contained 5% Acrylamide/N,N′-Methylenebisacrylamide (19:1), 0.05% Compound I, and 50% glycerol (Sigma) in 0.09 M sodium phosphate buffer (pH 6.8). Prior the photopolymerization, 0.4 ul of 0.04% aqueous solution of methylene blue and 1.2 ul of TEMED were added to 100 ul of the polymerization mix. The resulting mix was vortexed for 10-15 seconds and degassed for 3 minutes. The polymerization chamber was then filled with the polymerization mix and illuminated under Oriel Light Source (Oriel Instruments, Stratford, Conn., USA) for 15 minutes. After photopolymerization, the slide with the formed polymer template (matrix) was separated from the mask. The matrix was briefly washed with water and dried.

Providing Methacrylate Groups to Gel Array Templates

Gel array templates with were treated with Compound II aqueous solution in 0.1 M borate buffer (pH 8.0) for 1 hour, washed with MilliQ water for 5 minutes and dried. Next, the gel array templates were incubated for 1 min in Repel-Silane (Pharmacia), washed briefly with ethanol and MilliQ water and dried.

Loading of Oligonucleotide Solutions onto Gel Array Templates

Aqueous solutions of oligonucleotides with 5′-methacrylate groups in were dissolved in 0.1 M phosphate buffer (pH 7.2) with 19% glycerol and applied onto gel array templates by Quadrat II Robot (Argonne National Laboratory).

Graft Immobilization of Oligonucleotides on Gel Array Templates With Compound I

After oligonucleotides were delivered on to the gel array templates with Compound I, they illuminated under Oriel Light Source (Oriel Instruments, Stratford, Conn., USA) for 5 minutes. Next, the biochips were then briefly washed with MilliQ water, washed in 0.1×SSPE buffer with 0.05% SDS for 1 h at 60° C., washed briefly with MilliQ water and air dried.

Hybridization

The hybridization was carried out in 20-μl CoverWell chambers (Grace Biolabs) for 24 hours at room temperature, in a buffer containing 1 M guanidine thiocyanate (Fisher Scientific), 50 mM HEPES (pH 7.5) (Sigma), 10 mM EDTA (Ambion), 2 ug/μl BSA (Sigma), and 50 μg/μl salmon sperm DNA (Amersham-Pharmacia-Biotech). Prior to hybridization, the hybridization mix was filtered using 0.45-μm Ultrafree-MC Durapore low-binding centrifugation filter units (Millipore) by centrifugation at 6,000 rpm for 2 min at Eppendorf 5415C microcentrifuge (Eppendorf).

After hybridization, biochips were washed for 5 minutes in 6×SSPE (Sigma) containing 0.1% Triton X100 (Sigma) and were then briefly washed with MilliQ water twice. Next, biochips were dried at room temperature, and hybridization signals were acquired on Portable Biochip Reader (Argonne)

Documents

The following are incorporated by reference to the extent that they relate to materials or methods disclosed herein.

Printed Publications:

Campas M., Katakis I. 23 Trends in Anal. Chem., 49 (2004).

Christinsen C. B. V. 56 Talanta, 289 (2002).

Predki P. F. 8 Current opinion in Chemical Biology, 8 (2004).

Beaucage S. L. 8 Current medicinal Chemistry, 1213 (2001).

Kusnezow W., Hoheisel J. D. 33 BioTechniques, S14 (2002).

Talapatra A. et al. 3 Pharmacogenomics, 1 (2002).

Beattie W. G. et al. 4 Molec. Biotech., 213 (1995).

Rogers Y.-H. et al. 266 Anal. Biochem., 23 (1999).

Joos B. et al. 247 Anal. Biochem., 96 (1997).

Guo Z. et al. 22 Nucl. Acids Res., 5456 (1994).

Chrisley L. A. et al. 24 Nucl. Acids Res., 3031 (1996).

Boncheva M. et al. 15 Langmuir, 4317 (1999).

Khrapko K. R. et al. 1 DNA Sequence, 375 (1991).

Raddatz S. et al. 30 Nucl. Acids Res., 4793 (2002).

Kumar P., Gupta K. C. 14 Bioconjugate Chemistry, 507 (2003).

Yousaf M. N., Mrksich M. 121 J. Am. Chem Soc., 4286 (1999).

Coffinier Y. et al. 21 Langmuir, 1489 (2005).

Soellner M. B. et al. 125 J. Am. Chem. Soc., 11790 (2003).

Houseman B. T., Mrksich. 9 Chemistry & Biology, 443 (2002).

Devaraj N. K. et al. 127 J. Am. Chem. Soc., 8600 (2005).

Charles P. T. et al. 19 Langmuir, 1586 (2003).

Yershov G. et al. 93 Proc. Natl. Acad. Sci. USA, 4913 (1996).

Kelly J. J. et al. 311 Anal. Biochem, 311 (2002).

Patents

U.S. Pat. No. 6,692,972 B1.

U.S. Utility patent application Ser. No. 11/066,791 (Feb. 25, 2005).

We claim:

1. A method for immobilizing a biomolecule on a solid support the method comprising the steps of:
(a) providing a solid support comprising a first acrylate functional group;
(b) covalently attaching to the biomolecule a second acrylate functional group; and
(c) linking the first acrylate finctional group to the second acrylate functional group with a covalent bond.

2. The method according to claim 1, wherein the first acrylate functional group is a methacrylate functional group.

3. The method according to claim 1, wherein the second acrylate functional group is a methacrylate functional group.

4. The method according to claim 1, wherein the first and the second acrylate functional groups are methacrylate functional groups.

7. The method according to claim 1, wherein the providing step includes reacting a solid matrix with a compound of the formula:
H₂C═C(R¹)CO—X; wherein R¹ is hydrogen or alkyl; and X is a leaving group.

6. The method according to claim 5, wherein the leaving group is selected from the group consisting of N-hydroxysuccinimide and analogs and derivatives thereof, halides, and carboxylates.

7. The method according to claim 1, wherein the providing step includes reacting a solid matrix with a compound of the formula:
H₂C═C(R¹)CO—Y-Q-Y-Z; wherein R¹ is hydrogen or alkyl; Y=—NH— or —O—; Q is selected from the group consisting of alkylenes and poly(alkylene oxides); and Z is an activating group.

8. The method according to claim 7, wherein Q is alkylene of the formula (CH₂)ₙ, where n=4-12.

9. The method according to claim 7, wherein Q is PEG₄₀₀₋₆₀₀₀.

10. The method according to claim 7, wherein Z is selected from the group consisting of imidazolyl, N-hydroxysuccinimidyl, and analogues and derivatives thereof, halides, and carboxylates.

11. The method of claim 7, wherein Z is N-hydroxysuccinimidyl-COCH₂.

12. A method for providing a solid support comprising a methacrylate functional group, the method comprising the step of reacting a solid matrix with an amine of the formula:
H₂C═CH—R—NH₂; wherein R is selected from the group consisting of (CH₂)ₙ, where n=4-12, and PEG₄₀₀₋₆₀₀₀.

13. The method of claim 12, further comprising the step of subsequently reacting the aminated solid support with a methacrylating reagent of the formula:
H₂C═C(CH₃)CO—X; wherein X is selected from the group consisting of N-hydroxysuccinimidyl and analogs and derivatives thereof, halo, and carboxylates.

14. The method of claim 1, wherein the solid support further comprises a solid matrix selected from the group consisting of glass, quartz, plastics, silicon, hydrogel, nanoparticles, and a carbon nanotube.

15. The method according to claim 1, wherein the providing step includes (i) reacting a gold solid support with a compound of the formula R₁S—(CH₂)ₙ—NH₂, where n=4-12; and R₁ is selected from the group consisting of H, alkyl C₂-C₆—S, or disulfide; and (ii) subsequently reacting the gold solid support with a compound of the formula H₂C═C(CH₃)CO—X; where X is a leaving group.

16. The method according to claim 15, wherein the leaving group is selected from the group consisting of N-hydroxysuccinimidyl and analogs and derivatives thereof, halide, and carboxylates

17. The method according to claim 3, wherein the attaching step includes reacting the biomolecule with a compound of the formula: 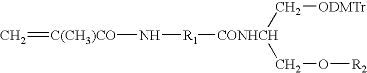
wherein R₁=(CH₂)ₙ, n=2-12; and R₂=P(OCH₂CH₂CN)N(iPr)₂ or CO(CH₂)ₘCO-CPG, where m=1-8.

18. The method according to claim 1 where the attaching step includes reacting the biomolecule with a compound of the formula:
H₂C═C(CH₃)CONH—(CH₂)ₙ—CONHS; where n=2-12.

19. The method according to claim 17 wherein the biomolecule is a biomolecule complex.

20. The method according to claim 1 where attaching step includes reacting the biomolecule with a compound of the formula:
H₂C═C(CH₃)CONH—(CH₂)ₙ—C₆H₄—COCH₂Br; where n=2-6.
or
H₂C═C(CH₃)CONH—R₃—NHCOCH₂—X; where X=Br, I; R₃ is (C₂-C₁₂)alkylene, or (C₃-C₈)cycloalkylene.

21. The method according to claim 1, wherein the biomolecule is a nucleic acid, and the second acrylate functional group is covalently attached to the biomolecule at a location selected from the group consisting of the 3′ end, and the 5′ end, of the biomolecule.

22. The method according to claim 1, wherein the biomolecule is a peptide or protein, and the second acrylate functional group is covalently attached to the biomolecule at the N-terminus of the biomolecule.

23. The method of claim 1, wherein the linking step includes covalent bond formation using radiation.

24. The method of claim 1, wherein the linking step includes covalent bond formation using a radical initiator.

25. A microarray comprising the reaction products of:
(a) a solid support comprising a first acrylate group; and
(b) a biomolecule comprising a second acrylate group;
where the reaction includes the attachment of one or more biomolecules to a solid support.
